(19) United States
(12) Patent Application Publication
(10) Pub. No.: US 2002/0151787 A1
(43) Pub. Date: Oct. 17, 2002

(54) Method of tumor imaging

(76) Inventors: Atle Bjornerud, Oslo (NO); Lars Johansson, Uppsala (SE)

Correspondence Address: Amersham Biosciences Corp., 800 Centennial Avenue, Piscataway, NJ, 08855, US

(21) Appl. No.: 10/094,442

(22) Filed: Mar. 8, 2002

Related U.S. Application Data
(63) Continuation of application No. PCT/NO00/00296, filed on Sep. 11, 2000.
(60) Provisional application No. 60/171,379, filed on Dec. 22, 1999.

(30) Foreign Application Priority Data
Sep. 13, 1999 (GB) 9921579.0
Mar. 31, 2000 (GB) 0007871.7

(51) Int. Cl. A61B 5/05

(52) U.S. Cl. 600/420

(57) ABSTRACT

A method of contrast-enhanced MR imaging to detect abnormal microvasculature, administering a superparamagnetic iron oxide blood pool magnetic resonance imaging contrast agent into the vasculature of a human or vascularized non-human body, generating T₁- and/or T₂ and T₂*-weighted magnetic resonance images of at least part of the said body into which said agent distributes.

This invention relates to improvements in and relating to magnetic resonance (MR) imaging of tumors, and in particular to the use of superparamagnetic iron oxides (SPIOs) in T₁- and/or T₂ and T₂* weighted MR imaging of tumors.

The early gadolinium chelate MR contrast agents, i.e. low molecular weight water soluble chelates such as gadopentetate (Magnevist from Schering) and gadodiamide (Omniscan from Nycomed Amersham), if administered into the vasculature, rapidly distribute into the extracellular space (i.e. the blood and the interstitium) and also are cleared relatively rapidly from the body, their contrast effect dropping almost exponentially with a half life of the order of 30 minutes.

SPIO blood pool MR contrast agents on the other hand are retained within the vasculature until eliminated through the Kupffer cells in the liver and may retain a prolonged contrast effect in the blood for a period of hours.

We have now found that such SPIO blood pool MR contrast agents may be used to detect capillary permeability abnormalities, including those occurring in tumors and inflammatory diseases.

Thus viewed from one aspect the invention provides a method of contrast-enhanced magnetic resonance imaging to detect abnormal microvasculature, said method comprising administering a superparamagnetic iron oxide blood pool magnetic resonance imaging contrast agent into the vasculature of a human or vascularized non-human (e.g. mammalian, avian or reptilian) body, generating T₁- and/or T₂ and T₂* weighted magnetic resonance images of at least part of the said body into which said agent distributes.

A preferred aspect of the invention provides a method of contrast-enhanced magnetic resonance imaging wherein the method is for detection of abnormal blood vessel wall permeability, microvascular density and/or microvascular composition.

Another preferred aspect of the invention provides a method of contrast-enhanced magnetic resonance imaging to detect abnormal blood vessel wall permeability, said method comprising the step of generating T₁-weighted magnetic resonance images of at least part of the said body into which said agent distributes and identifying regions of increased MR signal enhancement of tissue.

Regions of increased MR signal enhancement of tissue (as opposed to of veins or arteries large enough to be visualised in the MR image) will correspond to regions in which capillary wall permeability is higher than normal (i.e. the capillary walls are “leaky” for example as a result of angiogenesis). Since the capillary volume is typically only 3 to 10% of tissue volume, leaky regions of a tumor will show up as hyperintense in T₁-weighted MR images due to the SPIO contrast agent exerting its T₁-reducing affect over a much larger volume than in the “non-leaky” regions of a tumor.

Yet another preferred aspect of the invention provides a method for monitoring tumor microvascular density and/or microvascular composition, said method comprising administering into the vasculature of a patient, a SPIO blood pool MR contrast agent, and generating a T₂- and T₂*-weighted MR image of said tumor prior to any substantial leakage of the MR contrast agent.

Since increased capillary wall permeability in tumors has been found to have a positive correlation with tumor malignancy, use of the method of the invention to detect regions of hyperintensity due to increased capillary wall permeability is clearly of benefit to the diagnostician. Moreover grading of tumor angiogenesis may be of importance in tumor staging prognostication and treatment planning.

Likewise, use of the method of the invention to detect regions of hyperintensity due to angiogenesis may be used to allow the physician to monitor the success or otherwise of tumor treatment using angiogenesis inhibiting drugs, such as for example IM862, SU5416, Angiostatin etc.

Viewed from a further aspect the invention provides a method of monitoring therapeutic treatment, preferably tumor treatment and specially monitoring tumor treatment with angiogenesis inhibiting drugs. Said method comprises administration into the vasculature of a patient, who is receiving angiogenesis inhibiting drug treatment for a tumor, of a SPIO blood pool MR contrast agent, and generating a T₁-weighted MR image of said tumor and detecting regions of hyperintensity in said image attributable to increased capillary wall permeability (e.g. due to angiogenesis) at said tumor, said method preferably being repeated at intervals (e.g. of days or weeks) whereby to monitor changes in the extent of said regions of hyperintensity. This method may be used in screening of drugs for angiogenesis inhibiting properties, or for tumor staging or treatment planning.

Yet another preferred aspct of the invention provides a method for monitoring tumor therapy treatment, said method comprising administering into the vasculature of a patient receiving drug tumor treatment, a SPIO blood pool MR contrast agent, generating a T₂-T2* weighted MR image of said tumor and detecting regions of altered capillary density or microvascular composition, said method preferably being repeated at intervals whereby to monitor changes in the extent of said regions of hyperintensity.

In the methods of the invention, any solid tumor treatment may be monitored, e.g. metastatic disease and especially for mammary, prostate, bone and colorectal cancer.

The invention permits non-invasive detection of angiogenesis. Thus viewed from a further aspect the invention provides a method for the non-invasive detection of angiogenesis in a human or non-human vascularized subject, said method comprising administering a superparamagnetic iron oxide blood pool magnetic resonance imaging contrast agent into the vasculature of a human or vascularized non-human (e.g. mammalian, avian or reptilian) body and generating T₁-weighted magnetic resonance images of at least part of the said body into which said agent distributes whereby to detect regions of angiogenesis therein.

Viewed from a yet further aspect, the invention provides the use of a superparamagnetic iron oxide for the manufacture of a contrast medium for use in a method of diagnosis involving a method according to the invention.

In the methods of the invention, the SPIO blood pool MR contrast agent is preferably administered in a dose of 0.5 to 8 mg Fe/kg bodyweight, more preferably 1 to 6 mg Fe/kg, especially 2 to 5 mg Fe/kg. Desirably the contrast agent is injected or infused as a bolus over a period of 3 minutes or less, preferably 100 seconds or less (e.g. 15 to 70 seconds), still more preferably less than 60 seconds, especially 0.3 to 10 seconds. Contrast medium injection rates will desirably be in the range 0.01 to 10 mL/sec (e.g. 0.1 to 0.3 mL/sec) and more especially 0.3 to 3 mL/sec. The bolus should desirably be as tight as possible, e.g. by use of a power injector, and may be sharpened by the use of a physiological saline chaser. Administration may be into a vein or artery.

The SPIO blood pool MR contrast agent used according to the invention may be any physiologically tolerable agent comprising superparamagnetic iron oxide (or doped iron oxide) particles which has a blood half life (measured for example in the pig) of at least 10 minutes, preferably at least 30 minutes, more preferably at least 1 hour. Generally the contrast agent will be a particulate material having a particle size of 1 to 8000 nm, preferably 5 to 500 nm. Blood residence times for SPIOs can be enhanced by provision of an opsonization inhibiting coating, e.g. polyalkylene oxides (e.g. PEG), glycosaminoglycans (e.g. heparin or heparinoids, dermatan, hyaluronic acid, keratan, chondroitin, etc.). SPIOs having a r₂/r₁ ratio of less than 2.3, particularly less than 2.0, are especially preferred. Particularly suitable as SPIO agents are dextran or carboxy-dextran-coated SPIOs, the degraded starch coated SPIOs of WO97/25073 (preferably also provided with a PEG coating), AMI 7228 and the particulate agents described in WO95/05669, WO91/12526, WO91/12025, WO90/01899, WO88/00060, WO92/11037 and WO90/01295.

The SPIO agents are especially preferably members of the subclass known as ultra small superparamagnetic iron oxides (USPIO). The superparamagnetic agent is preferably a water-dispersible material comprising magnetic iron oxide particles having on their surfaces (e.g. as a coating), an optionally modified carbohydrate or polysaccharide or derivative thereof, e.g. a glucose unit containing optionally modified polysaccharide or derivative thereof, preferably an optionally modified dextran or starch or derivative thereof, for example a cleaved (e.g. oxidatively cleaved) starch or carboxylated dextran. Such iron oxide complexes preferably also comprise a further material (e.g. coating material), especially one which inhibits opsonization, e.g. a hydrophilic polymer, preferably a functionalized polyalkylene oxide, more preferably a functionalized polyethylene glycol (PEG), in particular methoxy PEG phosphate (MPP).

The iron oxide complexes preferably have a core (i.e. iron oxide particle) diameter (mode diameter) of 1 to 15 nm, more preferably 2-10 nm, especially 3-7 nm, a total diameter (mode particle size) of 1 to 100 nm, more preferably 5-50 nm, especially preferably 10-25 nm, an r₂/r₁ ratio at 0.47T and 40□C of less than 3, more preferably less than 2.3, still more preferably less than 2.0, especially preferably less than 1.8. The saturation magentization (Msat) at 1T is preferably 10 to 100 emu/gFe, more preferably 30-90 emu/gFe. One such agent currently undergoing clinical trials is known as Clariscan™ (Nycomed Imaging AS).

By a blood pool MR agent it is meant that the contrast agent remains within the vasculature and does not equilibrate within the ECF as a whole, i.e. unlike the small water-soluble gadolinium chelate ECF agents it does not extravasate except where vascular wall integrity is compromised, i.e. where vessel wall permeability is increased, e.g. where the vessels are “leaky”.

The SPIOs may be formulated for use in the method of the invention with conventional pharmaceutical carriers and excipients. Typically they will be in aqueous dispersion form, e.g. at an iron content of 10 to 50 mg Fe/mL, preferably 20 to 40 mg Fe/mL. Excipients that may be present include pH modifiers, chelating agents, viscosity modifiers, osmolality modifiers, etc.

Besides tumors, inflammatory and related diseases (such as atherosclerosis and rheumatoid arthritis) may compromise blood vessel wall permeability and regions of signal hyperintensity not associated with tumors may derive from such conditions. Likewise the technique may be used for therapeutic monitoring of rheumatoid disease, transplant rejection, ischemia, endometriosis etc.

The MR imaging technique used in the methods of the invention may be any one capable of generating T₁-weighted images, e.g. T₁-weighted spin echo (SE), fast spin echo, spoiled or non-spoiled 2D or 3D gradient echo, echo planar imaging or any hybrid of such sequences. Conventional spin echo techniques may be used; however if the dynamics of contrast enhancement are to be studied it is preferred to use a technique having an image acquisition time of 5 seconds or less, preferably 1 second or less, e.g. echo planar imaging, 2D or 3D-FLASH.

Regions of abnormal blood vessel wall permeability may be emphasised in the T₁-weighted images by subtracting equivalent non-contrast enhanced images. Likewise, regions of increased capillarization may be distinguished from regions of leaky blood vessels by subtraction of post contrast images, preferably one being after at least 45 minutes and the other being a first pass image.

In a further aspect of the invention, T₂-dependent sequences may also be used for tumour assessment, whereby the iron oxide nanoparticle causes signal reduction due to accumulation in macrophages. Increased macrophagic activity is often associated with inflammation and infection and angiogenesis.

Furthermore, T₂-depended sequences may be used to assess tumour vascularity prior to substantial contrast agent leakage into the tumour interstitium. Given that the signal change caused by the iron oxide nanoparticles is directly related to relative blood volume, the T₂ (or T₂*)-effect caused by the agent can be used to directly probe the relative blood volume or change in blood volume in response to therapy.

The methods of the invention will now be illustrated further with reference to the following non-limiting Examples and the accompanying drawings in which:

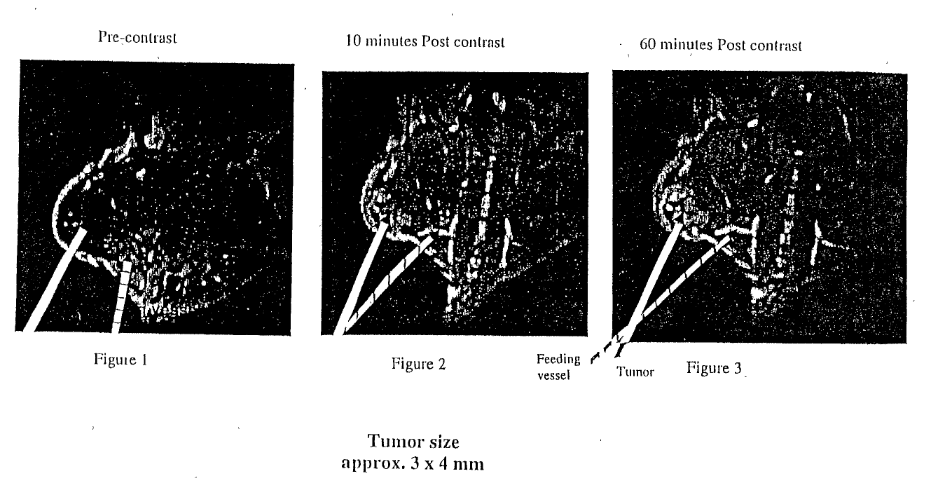

FIGS. 1 to 3 are pre and post contrast T₁-weighted MR images of a tumor implanted in the mouse leg.

An aqueous suspension of a SPIO blood pool MR contrast agent prepared according to the description in Example 12 of WO97/25073 was used in this Example. The characteristics of this suspension were: [Fc]=30.2 mg Fe/mL; density 1.0589 g/mL; r₁=19.3 s⁻¹ mM⁻¹; r₂=31.2 S⁻¹ mM⁻¹; r₂/r₁=1.61 (at 20 MHz and 37□ C.); saturation magnetization (Msat)=84 emu/g Fe.

### EXAMPLE 1

Tumor Imaging

Human colon cancer cells (LS174T) were implanted in the hind leg of a nude mouse and a tumor was allowed to grow.

5 mg Fe/kg bodyweight of the SPIO contrast medium was administered into a tail vein of the nude mouse over a period of 5 seconds.

T₁-weighted MR images of the same region of the tumor were recorded pre-contrast (FIG. 1), 10 minutes post contrast (FIG. 2) and 60 minutes post contrast (FIG. 3). The MR images were recorded on a 1.5T Philips Gyroscan NT MR imaging apparatus using a 3D-FFE sequence. [TR/TE/Flip angle=20 ms/3.6 ms/50□, FOV=60×60 mm, slice thickness 0.4 mm]

As can be seen, there was a rapid signal enhancement of visualizable vasculature in the tumor (dotted arrow) and a slower enhancement of a second part of the tumor (solid arrow). The first enhancement was clearly of a blood vessel rather than of the tumor tissue while the second enhancement was of tumor tissue arising as a result of slow leakage of contrast agent into the interstitium.

1. A method of contrast-enhanced magnetic resonance imaging to detect abnormal microvasculature, said method comprising administering a superparamagnetic iron oxide blood pool magnetic resonance imaging contrast agent into the vasculature of a human or vascularized non-human body, generating T₁- and/or T₂ and T₂*-weighted magnetic resonance images of at least part of the said body into which said agent distributes.

2. A method according to claim 1 of contrast-enhanced magnetic resonance imaging wherein the method is for detection of abnormal blood vessel wall permeability, microvascular density and/or microvascular composition.

3. A method according to any of claims 1 to 2 of contrast-enhanced magnetic resonance imaging to detect abnormal blood vessel wall permeability, said method comprising the step of generating T₁-weighted magnetic resonance images of at least part of the said body into which said agent distributes and identifying regions of increased MR signal enhancement of tissue.

4. A method according to any of claims 1 and 2 for monitoring tumor microvascular density and/or microvascular composition, said method comprising administering into the vasculature of a patient, a SPIO blood pool MR contrast agent, and generating a T₂- and T₂*-weighted MR image of said tumor prior to any substantial leakage of the MR contrast agent.

5. A claim according to claim 1 for monitoring of therapeutic treatment, said method comprising administering into the vasculature of a patient receiving drug treatment, a SPIO blood pool MR contrast agent, generating a T₁-weighted MR image and detecting regions of hyperintensity in said image attributable to increased capillary wall permeability, said method preferably being repeated at intervals whereby to monitor changes in the extent of said regions of hyperintensity.

6. A method according to any of claim 1 and 5 for monitoring tumor therapy treatment, said method comprising administering into the vasculature of a patient receiving drug tumor treatment, a SPIO blood pool MR contrast agent, generating a T₁-weighted MR image of said tumor.

7. A method for monitoring tumor treatment according to claim 6 wherein the said treatment is with angiogenesis inhibiting drugs.

8. A method according to claim 1 for monitoring tumor therapy treatment, said method comprising administering into the vasculature of a patient receiving drug tumor treatment, a SPIO blood pool MR contrast agent, generating a T₂-T2* weighted MR image of said tumor and detecting regions of altered capillary density or microvascular composition, said method preferably being repeated at intervals whereby to monitor changes in the extent of said regions of hyperintensity.

9. A method of claim 1 for the non-invasive detection of angiogenesis in a human or non-human vascularized subject, said method comprising administering a superparamagnetic iron oxide blood pool magnetic resonance imaging contrast agent into the vasculature of a human or vascularized non-human body and generating T₁-weighted magnetic resonance images of at least part of the said body into which said agent distributes whereby to detect regions of angiogenesis therein.

10. The use of a superparamagnetic iron oxide for the manufacture of a contrast medium for use in a method of diagnosis involving a method according to either of claims 1 to 9.
